(19) United States
(12) Patent Application Publication
(10) Pub. No.: US 2024/0325437 A1
(43) Pub. Date: Oct. 3, 2024

(54) METHODS AND COMPOSITIONS FOR USE IN IMPROVING ADAPTIVE IMMUNITY IN THE ELDERLY POPULATION

(71) Applicant: Technion Research & Development Foundation Limited, Haifa (IL)

(72) Inventors: Noga RON-HAREL, Haifa (IL); David EZUZ, Haifa (IL)

(73) Assignee: Technion Research & Development Foundation Limited, Haifa (IL)

(21) Appl. No.: 18/619,343

(22) Filed: Mar. 28, 2024

Related U.S. Application Data
(60) Provisional application No. 63/455,034, filed on Mar. 28, 2023.

(51) Int. Cl. A61K 33/26; A61K 31/49; A61K 38/38; A61K 39/39; A61P 37/04

(52) U.S. Cl. CPC A61K 33/26; A61K 31/49; A61K 38/385; A61K 39/39; A61P 37/04

(57) ABSTRACT

A method of increasing proliferation of adaptive immune cells of a subject at least 65 years old is provided. The method comprising contacting the immune cells with an agent which increases the level of labile iron in the immune cells and/or reduces toxicity of hemoglobin, heme or a degradation product thereof, thereby increasing the proliferation of the adaptive immune cells of the subject.

### RELATED APPLICATIONS

This application claims the benefit of priority under 35 USC § 119 (e) of U.S. Provisional Patent Application No. 63/455,034 filed on Mar. 28, 2023, the contents of which are incorporated by reference as if fully set forth herein in their entirety.

### SEQUENCE LISTING STATEMENT

The XML file, entitled 99140SequenceListing.xml, created on Mar. 28, 2024, comprising 11,242 bytes, submitted concurrently with the filing of this application is incorporated herein by reference.

### FIELD AND BACKGROUND OF THE INVENTION

The present invention, in some embodiments thereof, relates to methods and compositions for use in improving adaptive immunity in the elderly population.

Advanced age is a major risk factor for infections, autoimmunity, cancer, atherosclerosis, metabolic diseases, and impaired vaccine responses. Thus, the extension in life span creates a challenge to facilitate healthy aging. An aged/dysfunctional T cell immunity is sufficient to drive the senescence and aging of solid organs, and the development of age-related morbidity^{1,2}. Thus, restoring immune functions (and specifically T cell immunity) in the elderly holds the promise for a comprehensive and simultaneous targeting of multiple age-related pathologies.

Immune aging is characterized by loss of adaptive immunity and an increase in non-specific innate immunity (Inflammaging)^{3,4}. Amongst immune system components most affected by aging are T lymphocytes^{5,6}. These cells of the adaptive immune system play a central role in the immune defense against intruders, and support tissue homeostasis and function.

It is therefore imperative to understand the mechanisms that govern lymphocyte disfunction in the elderly, thus developing new therapeutic modalities for this growing population.

Additional background art includes:
Cummins et al. FASEB J. 2012 July; 26 (7): 2911-2918.
Li et al. Heliyon. 2017 May; 3 (5): e00303.
WO2012050874

### SUMMARY OF THE INVENTION

According to an aspect of some embodiments of the present invention there is provided a method of increasing proliferation of adaptive immune cells of a subject at least 65 years old, the method comprising contacting the immune cells with an agent which increases the level of labile iron in the immune cells and/or reduces toxicity of hemoglobin, heme or a degradation product thereof, thereby increasing the proliferation of the adaptive immune cells of the subject.

According to an aspect of some embodiments of the present invention there is provided a method of increasing efficacy of adaptive immune cells of a subject at least 65 years old, the method comprising contacting the immune cells with an agent which increases the level of labile iron in the immune cells and/or reduces toxicity of hemoglobin, heme or a degradation product thereof, thereby increasing the efficacy of the adaptive immune cells of the subject.

According to an aspect of some embodiments of the present invention there is provided a method of treating or preventing an infectious disease in a subject at least 65 years old, the method comprising administering to the subject a therapeutically effective amount of an agent which increases the level of labile iron in immune cells and/or reduces toxicity of hemoglobin, heme or a degradation product thereof, thereby treating or preventing the infectious disease in the subject at least 65 years old.

According to an aspect of some embodiments of the present invention there is provided an agent which increases the level of labile iron in immune cells and/or reduces toxicity of hemoglobin, heme or a degradation product thereof for use in treating an infectious disease in a subject at least 65 years old.

According to an aspect of some embodiments of the present invention there is provided a method of vaccinating a subject at least 65 years old, the method comprising administering to the subject a vaccine and an agent which increases the level of labile iron in immune cells and/or reduces toxicity of hemoglobin, heme or a degradation product thereof, thereby vaccinating the subject.

According to an aspect of some embodiments of the present invention there is provided an agent which increases the level of labile iron in immune cells and/or reduces toxicity of hemoglobin, heme or a degradation product thereof as an adjuvant for a vaccine in a vaccination treatment of a subject at least 65 years old.

According to an aspect of some embodiments of the present invention there is provided a method of increasing proliferation of adaptive immune cells of a subject in need thereof, the method comprising: contacting the immune cells with an agent which increases the level of labile iron in the immune cells and/or reduces toxicity of hemoglobin, heme or a degradation product thereof, thereby increasing the proliferation of the adaptive immune cells of the subject, wherein the subject has been selected by determining a marker for dysfunctional T cells.

According to an aspect of some embodiments of the present invention there is provided a method of treating or preventing an infectious disease or an inflammatory disease in a subject at least 65 years old, the method comprising administering to the subject a therapeutically effective amount of an agent which increases the level of labile iron in immune cells and/or reduces toxicity of hemoglobin, heme or a degradation product thereof, thereby treating or preventing the infectious disease or an inflammatory disease in the subject at least 65 years old.

According to an aspect of some embodiments of the present invention there is provided a method of treating or preventing an infectious disease or an inflammatory disease in a subject, the method comprising: (a) selecting a subject exhibiting a level of dysfunctional T cells above a predetermined threshold; (b) administering to the subject an agent which increases the level of labile iron in immune cells and/or reduces toxicity of hemoglobin, heme or a degradation product thereof, thereby treating or preventing the infectious disease or inflammatory disease in the subject.

According to an aspect of some embodiments of the present invention there is provided a method of vaccinating a subject at least 65 years old, the method comprising administering to the subject a vaccine and an agent which increases the level of labile iron in immune cells and/or reduces toxicity of hemoglobin, heme or a degradation product thereof, thereby vaccinating the subject.

According to an aspect of some embodiments of the present invention there is provided a method of vaccinating a subject in need thereof, the method comprising: (a) selecting a subject exhibiting a level of dysfunctional T cells above a predetermined threshold; (b) administering to the subject a vaccine and an agent which increases the level of labile iron in immune cells and/or reduces toxicity of hemoglobin, heme or a degradation product thereof, thereby vaccinating the subject.

According to some embodiments of the invention, the cells are lymphocytes.

According to some embodiments of the invention, the lymphocytes are T lymphocytes.

According to some embodiments of the invention, the lymphocytes are B lymphocytes.

According to some embodiments of the invention, the vaccine is selected from the group consisting of a flu vaccine, a pneumococcal vaccine, a shingles vaccine, and a tetanus-diptheria-pertussis vaccine (Tdap).

According to some embodiments of the invention, the agent is at least one selected from the group consisting of:
(i) labile iron;
(ii) a scavenger of hemoglobin or heme or a degradation product thereof;
(iii) a lysosomal inducer; and
(iv) a heme crystal breaking agent.

According to some embodiments of the invention, the labile iron is selected from the group consisting of ferric ammonium citrate, Ferrous sulfate, ferrous gluconate, ferrous ascorbate, ferrous fumarate, ferrous bisglycinate, iron polymaltose, iron sucrose and Iron Dextran.

According to some embodiments of the invention, the scavenger is selected from the group consisting of hemopexin, heptoglobin and albumin.

According to some embodiments of the invention, the lysosomal inducer is selected from the group consisting of ambroxol, gastrodin, a CDK4/6 inhibitor and an autophagy inducer.

According to some embodiments of the invention, the CDK4/6 is selected from the group consisting of LY2835219 (e.g., Abemaciclib) and PD0332991 (e.g., palbociclib).

According to some embodiments of the invention, the autophagy inducer is selected from the group consisting of rapamycin, torin1, metformin, resveratrol and spermidine.

According to some embodiments of the invention, the heme crystal breaking agent is selected from the group consisting of quinine, chloroquine, amodiaquine, quinidine, quinacrine, halofantrine and mefloquine.

According to some embodiments of the invention, the agent is administered or formulated for administration in an acute manner (i.e., not more than 14 days).

According to some embodiments of the invention, the scavenger is administered or formulated for administration in a chronic manner (i.e., more than 2 weeks).

According to some embodiments of the invention, the subject is selected according to measuring a level of protein or mRNA of at least one stress gene selected from the group consisting of CD39, HO1, HBB-BS, HBA, HMOX1, HEBP1, BLVRB, CPOX, FTL1, HMBS and FTH1, wherein a level above a predetermined threshold (e.g., higher than that of a young subject below 65 years old) is indicative of suitability to the treatment.

According to some embodiments of the invention, the level is a peripheral blood.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-J: The aged spleen microenvironment induces T cell dysfunction. (A) experimental design. Naïve CD4⁺ T cells were purified from the spleen and lymph nodes of aged mice and activated ex vivo using plate bound anti-CD3/anti-CD28 for 24 or 48 hr prior to analysis by flow cytometry, to quantify: (B) cell viability, (C) expression of the early activation marker CD69, and (D) proliferation. In parallel experiments, pure naïve CD4⁺ T cells were extracted from young and aged mice (spleen and lymph nodes (LNs) and analyzed by flow cytometry to quantify CD39 expression (E). (F) T cells derived from the spleen and lymph nodes of mice at different ages were analyzed by flow cytometry to assess population composition. CM: central memory; EM: effector memory. (G) Experimental design. Young T cells derived from td-Tomato-transgenic mice were transferred (i.v.) to young or aged C57bl/6 recipients. The spleen and LNs of recipient mice were collected after 2 weeks, CD4+ T cells were purified, activated ex vivo for 48 hr, and analyzed by flow cytometry to assess proliferation (H). Additional analyses (2-3 weeks following cell transfer CD3⁺ T cells purification from the spleen and lymph nodes for analysis of cell size (I) and CD39 expression (J) by flow cytometry. (*P<0.05, **P<0.01, ***P<0.001, ****P<0.0001; student's t-test).

FIGS. 2A-L: T cells in the aged spleen are enriched with proteins associated with stress and inflammation. (A) Experimental design. Naïve CD4⁺ T cells were purified from the spleens of young and aged mice. The cells were either activated for 24 hr ex vivo or directly processed for protein extraction and digestion prior to proteomics analysis by liquid chromatography/mass spectrometry. (B) Principal component analysis. (C) Volcano plot showing differences in protein levels between activated young and aged T cells. Green signifies statistical significance. (D) Pathways enriched among proteins significantly overrepresented in young vs aged T cells following activation. (E) Volcano plot showing differences in protein levels between naive young and aged T cells. Green signifies statistical significance. (F) Pathways enriched among proteins significantly overrepresented in aged vs. young naïve T cells. (G) Venn Diagram showing overlap between proteins overexpressed in aged naïve T cells compared to young and proteins naturally inducing with T cell activation. (H) Heatmap summarizes proteins associated with heme metabolism and detoxification that are significantly overrepresented in aged naïve T cells compared to young. (I) Key proteins involved in heme detoxification. (J-L) Differences in expression of HO-1 and ferritin (FTH) in naïve CD4⁺ T cells were validated by flow cytometry. (*P<0.05, **P<0.01, ***P<0.001, ****P<0.0001; student's t-test).

FIGS. 3A-L: Heme and the products of its degradation are accumulating in aged spleens.

Spleens derived from young and aged mice were used for quantifying heme content in spleen extract (extracellular fluids collected during cell separation; A) and intracellularly, in bulk CD3+ T cells (B) using a colorimetric assay. (C) Total and unconjugated bilirubin levels in spleen extract of young and aged mice. (D) Left panels—Histology. Paraffin sections of spleens derived from aged and young mice, with H&E staining. RP—red pulp; WT—white pulp. Right panel—representative images of frozen spleen sections stained with anti-CD169 to mark marginal zone (MZ) macrophages. (E) Paraffin sections of spleens derived from aged and young mice, processed by Prussian Blue method to detect iron depositions in the spleen. (F) Experimental design. Young T cells derived from td-Tomato-transgenic mice were transferred (i.v.) to young or aged C57bl/6 recipients. The spleen and LNs of recipient mice were collected after 2 weeks and immediately analyzed by flow cytometry to quantify ferritin (FTH; G, H) and HO-1 (I, J). (K) Young T cells were cultured in the presence of spleen extract (SE)±an iron chelator, DFO. Cell viability was quantified by flow cytometry, using Zombie. (L) Representative flow plots. (*P<0.05, **P<0.01, ***P<0.001, ****P<0.0001; student's t-test).

FIGS. 4A-Z1: Heme and spleen extract (SE) drive aging-like phenotypes in young T cells. (A) Experimental design. CD3+ T cells derived from young mice were cultured in the presence of either heme or SE for 48 hr. In some experiments (B-E), cells were supplemented with IL-7 and kept in a resting state. In some experiments (F-M) cells were stimulated with plate bound anti-CD3 anti-CD28. Analysis was performed by flow cytometry. Heme reduces cell viability (B), and induces expression of ferritin (C), HO-1 (D), and CD39 (E) in a dose-dependent way. Bovine serum albumin (BSA) rescued T cells viability upon exposure to heme (F, G) or SE (H,I). BSA rescued T cells proliferation upon exposure to heme (J, K) or SE (L, M). (N) Immunophenotyping of T cells derived from young mice, following ex vivo incubation with and without hemc. CM—central memory; EM—effector memory. (O) Young and aged T cells were treated with increasing doses of RSL, a potent inducer of ferroptosis. Cell viability was analyzed after 48 hrs, demonstrating that aged T cells are significantly more tolerant to ferroptosis than young T cells. (P) A representative plot showing proliferation of cells treated with heme±SnPP, an HO-1 inhibitor. (Q) Analysis of CD39 expression levels in response to heme supplementation. (R,S) Analysis of CD39 expression levels in cells treated with heme±SnPP. (T, U) DCFDA was used to quantify ROS levels in young T cells cultured in media supplemented with hemc±BSA for 48 hr. (V-X) analysis of T cell viability (V-W) and proliferation (X) following culture in media supplemented with heme±NAC for 48 hr. (Y, Z) Analysis of BODIPY fluorescence intensity, as an indication of lipid peroxidation in young T cells treated with heme±BSA. (Z1) Analysis of lipid peroxidation using the liperfluo reagent in young and aged T cells. Bar graphs represent mean±SEM. Data points represent single mice (*P<0.05, **P<0.01, ***P<0.001, ****P<0.0001; student's t-test).

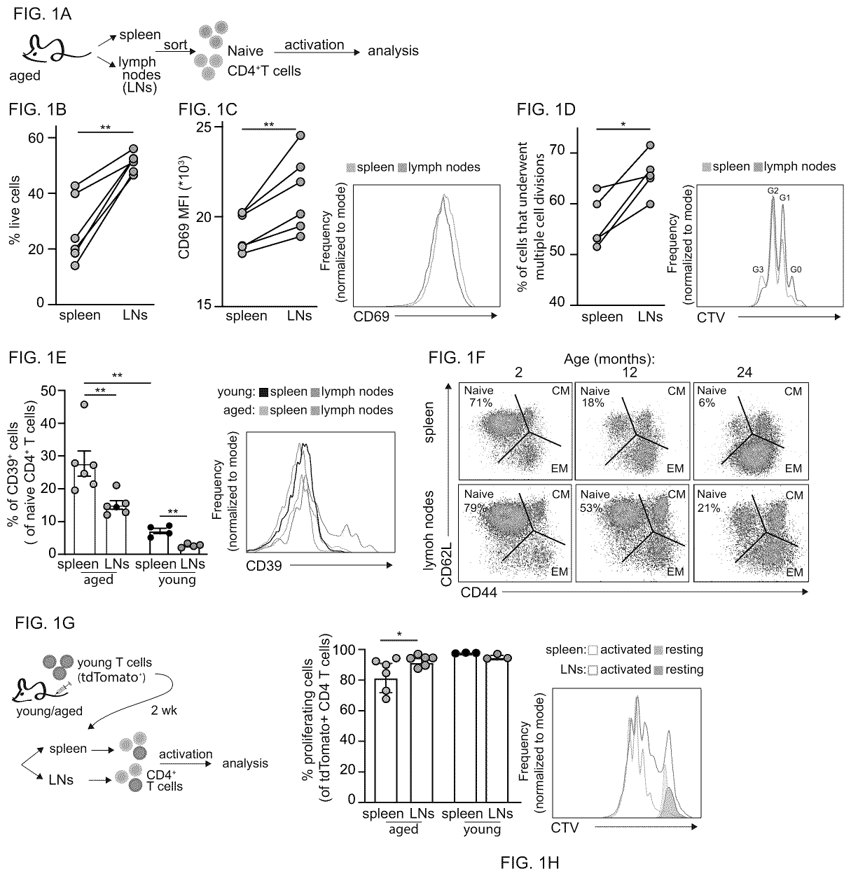

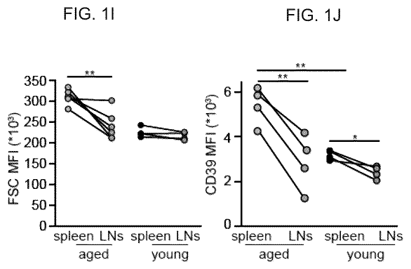

FIGS. 5A-J: Aged T cells are iron deficient. Young T cells were stimulated ex vivo using plate bound anti-CD3/anti-CD28. Cells were collected at different time points, up to 72 hr, post-activation,) and loaded with Ferro-Orange, for detection of labile iron. (A) a representative plot showing activation-induced increase in cellular labile iron in CD3⁺ T cells. (B) Analysis of the same data, separating Ferro-Orange intensity in young CD4⁺ vs young CD8⁺ T cells. (C) Kinetic changes in labile iron levels in young and aged T cells following activation. (D) a representative plot depicting differences in Ferro-Orange intensity at 72 hr post-activation. (E) Activation-induced differences in Ferro-Orange intensity in aged CD4⁺ vs aged CD8⁺ T cells. (F) Analysis of CD71 (transferrin receptor 1) expression levels in young and aged T cells, following activation. (G, H) Adoptive transfer experiment was performed as described in FIG. 1G. 2 weeks following transfer of TdTomato⁺ T cells, the spleens were extracted, CD3+ T cells purified, and cultured ex vivo with RSL3 to assess ferroptosis resistance. (G) Differences in ferroptosis resistance between Transferred (TdTomato⁺) and (H) endogenous (TdTomato⁻) CD4⁺ and CD8⁺ T cells. Line graphs represent mean±SEM. Data points (G,H) represent single mice. (*P<0.05, **P<0.01, ***P<0.001, ****P<0.0001; B-F: unpaired student's t-test, comparing the two datasets at each time point; G-H: paired student's t-test; I-J: unpaired student's t-test, comparing each condition to control cells).

FIGS. 6A-M: Iron supplementation rescued vaccination responses in aged T cells. (A) Ferro-Orange fluorescent intensity was quantified in young and aged T cells, and aged T cells supplemented with ferric ammonium citrate (FAC). (B) A representative plot shows intensity shift in labile iron content in aged T cells after supplementation with FAC. (C-E) young or aged T cells were loaded with CellTrace Violet and stimulated ex vivo with and without supplementation of FAC or holo transferrin, prior to analysis of proliferation. (C, D) analysis of aged T cells. (E) analysis of young T cells. (F) Activation-induced changes in mitochondrial iron pools comparing young and aged T cells, quantified using mito-ferro green. (G) Quantification of mitochondrial labile iron in aged T cells at 24 hr post-activation±FAC. (H) A representative plot depicting changes in proliferation in aged T cells supplemented with FAC±a FECH inhibitor that prevents iron incorporation in the last step of heme biosynthesis. (I) Scheme of experimental design. Aged C57B1/7 wild type mice were inoculated with transgenic T cells bearing a known antigen specificity against ovalbumin (OVA). Each mouse was injected with a 1:1 mixture of OTII (TdTomato⁺CD4⁺) and OTI (CD45.1⁺CD8⁺) T cells. After two weeks, recipient mice were vaccinated with OVA/adjuvant intraperitoneally. Control mice received saline. On days 1 and 3 post-vaccination, vaccinated mice received i.v. infusions of FAC or saline. The mice were sacrificed on day 5 post-vaccination and T cell content in the spleen was analyzed. (J, K) Quantitation (J) and a representative plot (K) showing percentage of OTII⁺ T cells. (L,M) Quantitation (L) and a representative plot (M) showing percentage of OTI⁺ T cells. Line graphs represent mean±SEM. Data points (A,G,L, K) represent single mice. (*P<0.05, **P<0.01, ***P<0.001; unpaired student's t-test).

FIGS. 7A-I: Heme induces iron deficiency by inhibiting lysosome activity. (A) T cell activation induces lysosomal biogenesis. Graph summarizes lysotracker MFI at different times post-activation. (B) lysosomal activity is increased with T cell activation. Lysosomal biogenesis was quantified by flow cytometry (C) and microscopy (D) in T cells+/−activation+/−heme. (E) analysis of lysosomal activity under the same conditions. (F) CD63, a marker of late endosomes, was quantified in young and aged T cells, by flow cytometry. (G) CD63 expression was quantified on T cells cultured with heme at different concentrations. (H) FOXO1 levels in young T cells exposed to heme or SE for 4-48 hr. (I) FOXO1 levels in young and aged T cells derived from spleens or LNs, as analyzed by flow cytometry. (*P<0.05, **P<0.01, ***P<0.001, ****P<0.0001; student's t-test).

FIG. 8: Aged T cells were activated ex vivo with and without quinine to assess its effect on proliferation.

FIGS. 9A-B: Quinine increases labile iron pools in aged T cells. (A) Intracellular free Iron measurement (detected by Ferro-Orange) in aged CD3⁺ T cells, cultured in the presence or absence of quinine (15 uM). (B) Quantitation of HO-1 levels in aged CD3⁺ T cells follow activation.

### DESCRIPTION OF SPECIFIC EMBODIMENTS OF THE INVENTION

The present invention, in some embodiments thereof, relates to methods and compositions for use in improving adaptive immunity in the elderly population.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors identified heme that is accumulating in the spleen of aged mice as a novel driver of T cell dysfunction with aging. Exposure to heme or to the aged spleen extracellular microenvironment (Interstitial-fluid enriched fraction of the spleen which may be referred to herein as “spleen extract” abbreviated as SE) induced T cell differentiation and depletion of the naïve T cell compartment, reduced T cell viability and impaired T cell proliferation. These defects were rescued by supplementing the cells with labile iron, treating them with heme scavengers, like serum albumin, subjecting the cells to heme crystal breaking agent (such as quinine). Mechanistically, T cells derived from aged spleens overexpress proteins mediating heme degradation (HO-1) and storage of extra iron (ferritin). The present data suggests that these defense mechanisms allow aged T cells to survive the hostile, aged microenvironment, at the cost of critical T cell functions, such as proliferation. Upon activation, lysosomal defects (partly induced by heme) are preventing the breakdown of iron stores (ferritin), depriving the cells from labile iron that is critical for T cell activation and proliferation. Based on this mechanism, the present inventors were able to show that iron supplementation rescues T cell proliferation and improves in vivo response to vaccination. Altogether, these results provide the basis for increasing the proliferative capacity of adaptive immune cells particularly in those of the elderly population being at least 65 years old or those which have dysfunctional immune cells.

Thus, according to an aspect of the invention there is provided a method of increasing proliferation of adaptive immune cells of a subject at least 65 years old, the method comprising contacting the immune cells with an agent which increases the level of labile iron in the immune cells and/or reduces toxicity of hemoglobin, heme or a degradation product thereof, thereby increasing the proliferation of the adaptive immune cells of the subject.

As used herein “increased” refers to an increase of at least 10%, 20%, 30%, 40%, 50%, 60%, 70%, 80%, 90%, 1.2 fold 1.5 fold, 2 fold, 5 fold 10 fold or more compared to that of a control sample. A control sample depends on the measured parameter and can be of the same subject prior to treatment; or of subjects which are characterized by dysfunctional innate immunity cells due to age or due to toxic spleen environment.

As used herein “proliferation” refers to cell expansion in response to an immunostimulant (anti CD3 and/or anti CD28). Expansion can be determined using any method known in the art such as but not limited to MTT, XTT, or by tracking cell proliferation by permanently binding cellular proteins and rendering the cell fluorescent. In a certain embodiment, this can be determined using commercial kits such as, but not limited to, trace violet proliferation kit (e.g., Invitrogen).

As used herein “adaptive immune cells” refers to cells which mediate an immune response that is characterized by specificity, immunological memory, and/or self/non-self recognition. The cells can be T lymphocytes (CD3+, e.g., CD4+ and/or CD8+) and/or B lymphocytes. According to a specific embodiment, the cells are CD4+ T cells.

According to a specific embodiment, the cells are lymphocytes.

According to a specific embodiment, the lymphocytes are T lymphocytes.

According to a specific embodiment, the lymphocytes are B lymphocytes.

Alternatively, or additionally, there is provided a method of treating or preventing an infectious disease in a subject at least 65 years old, the method comprising administering to the subject a therapeutically effective amount of an agent which increases the level of labile iron in immune cells and/or reduces toxicity of hemoglobin, heme or a degradation product thereof, thereby treating or preventing the infectious disease in the subject at least 65 years old.

Alternatively, or additionally, there is provided an agent which increases the level of labile iron in immune cells and/or reduces toxicity of hemoglobin, heme or a degradation product thereof for use in treating an infectious disease in a subject at least 65 years old.

As used herein “subject” refers to a mammal, preferably a human being, male or female. The subject can be healthy or diagnosed with any kind or morbidity (e.g., diabetes or cardiac disease) or is at risk thereof.

According to a specific embodiment, the subject is not diagnosed or does not exhibit symptoms of an infection (e.g., fever).

According to a specific embodiment, the subject is diagnosed or exhibits symptoms of an infection (e.g., fever).

According to a specific embodiment, the subject is at an age at which it is recommended by the CDC or other governmental agencies to get vaccinated to avoid adverse symptoms which typically increase with age and even cause an increase in mortality.

As used herein “an infectious disease” refers to any disease caused by a microorganism e.g., bacteria, virus, fungi or parasite. The infection can be a primary infection or a secondary infection.

Alternatively or additionally, there is provided a method of treating or preventing an inflammatory disease in a subject at least 65 years old, the method comprising administering to the subject a therapeutically effective amount of an agent which increases the level of labile iron in immune cells and/or reduces toxicity of hemoglobin, heme or a degradation product thereof, thereby treating or preventing the inflammatory disease in the subject at least 65 years old.

Alternatively or additionally, there is provided an agent which increases the level of labile iron in immune cells and/or reduces toxicity of hemoglobin, heme or a degradation product thereof for use in treating an inflammatory disease in a subject at least 65 years old.

As used herein “inflammatory disease” or “inflammation” is a medical condition in which there is basal secretion of pro-inflammatory cytokines at a level which is above that found in a healthy tissue. These include but are not limited to IL-6, TNFalpha and IL-1beta.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

Alternatively, or additionally, the subject may be of any age (at least 65 years old or younger) and is selected for treatment by measuring a marker which is associated with dysfunctional T cells.

According to another embodiment, the subject is characterized by an immunological age in which the innate immune cells are dysfunctional (as can be determined using a marker, as described below), regardless of the chronological age and therefore the subject can be below 65 years, e.g., 18-64.

According to a specific embodiment, the marker is a molecular marker.

According to some embodiments, the marker is a cell surface marker.

For example, measuring in T cells CD39 levels.

The name of the molecular marker is according to the gene symbol.

The present inventors have found that CD39 is induced in response to the aged spleen microenvironment, therefore it can be used as a sensor of the microenvironment inferring cellular iron deficiency. The present inventors have also shown that FTH, HO1, FTL1, FTH, BLVRB, BLVRA are overly expressed on dysfunctional T cells.

Thus, according to some embodiments of the invention, the subject is selected by measuring a level of protein or mRNA of at least one stress gene.

Any stress gene mentioned herein is mentioned by its gene symbol.

As used herein “stress gene” refers to a gene (also referred to as a marker) which expression at the mRNA and/or protein level is increased in immune cells as a result of exposure to the aged spleen environment which is characterized by the toxicity of hemoglobin, heme or a degradation product thereof.

According to some embodiments, the stress gene is selected from the group consisting of CD39, HO1, HBB-BS, HBA, HMOX1, HEBP1, BLVRB, CPOX, FTL1, HMBS and FTH1, wherein a level above a predetermined threshold (e.g., higher than that of a young subject below 65 years old) is indicative of suitability to the treatment (also referred to as a biomarker).

According to a specific embodiment, the stress gene is ferritin (FTL/H), HO-1 and/or CD39.

According to a specific embodiment, the stress gene is HO-1 and/or CD39. According to a specific embodiment, the stress gene is CD39.

According to some embodiments, the stress gene is selected from the group consisting of CD39, HBB-BS, HBA, HMOX1, HEBP1, BLVRB, CPOX, FTL1 and HMBS, wherein a level above a predetermined threshold (e.g., higher than that of a young subject below 65 years old) is indicative of suitability to the treatment

As used herein “A level above a predetermined threshold means” a statistically significant increase in T cells which overexpress the marker at the protein or mRNA level as compared to that of functional T cells.

It will be appreciated that the increase is with respect to the level of such cells in a subject in which the T cells are functional (as determined by stimulation ex vivo to assess proliferation).

Determination of expression at the protein level can be done using methods which are well known in the art. These include, but are not limited to immunodetection assays such as FACS, ELISA and western blot analysis, immunohistochemistry and the like, which may be effected using antibodies specific to the detected protein product.

Methods useful for monitoring the expression level of specific genes are well known in the art and include RT-PCR, semi-quantitative RT-PCR, Northern blot, RNA in situ hybridization, Alternatively, selection can be done by imaging. According to some embodiments, MRI can be used to quantify tissue iron content (Sorokin et al 2022, The American Journal of Human Genetics 109, 1092-1104).

As used herein “labile iron” refers to a form of iron that is available to the cells and does not require lysosomal activity for its lability.

As used herein “an agent which increases the level of labile iron in immune cells and/or reduces toxicity of hemoglobin, heme or a degradation product thereof” refers to a chemical or biological substance.

When referring to toxicity it may be manifested by an effect on cell proliferation in response to stimulation ex vivo to assess proliferation. According to some embodiments, cytotoxicity such as that of the degradation product affects mitochondrial activity in the cell.

According to a specific embodiment, the agent is at least one of:
(i) labile iron;
(ii) a scavenger of hemoglobin or heme or a degradation product thereof;
(iii) a lysosomal inducer; and
(iv) a heme crystal breaking agent.

As used herein, in the context of agents that can be used in accordance with the present teachings “labile iron” refers to an iron derivative that is available to the cells and does not require an active uptake through the transferrin receptor or a protein carrier.

Examples of labile iron agents include, but are not limited to those selected from the group consisting of ferric ammonium citrate (FAC), Ferrous sulfate, ferrous gluconate, ferrous ascorbate, ferrous fumarate, ferrous bisglycinate, iron polymaltose, iron sucrose and Iron Dextran.

These are commercially available from various vendors such as listed below. This is a non-exhaustive list. Ferric Ammonium Citrate (FAC):

Sigma-Aldrich (now part of MilliporeSigma), Alfa Aesar, Fisher Scientific; Ferrous Sulfate: GlaxoSmithKline (brand name: Ferrous Sulfate Tablets), Mylan Pharmaceuticals (brand name: Ferrous Sulfate Tablets), Nature Made (a brand of Pharmavite LLC); Ferrous Gluconate: Gericare Pharmaceuticals, Major Pharmaceuticals (brand name: Ferrous Gluconate Tablets); Ferrous Ascorbate: Abbott Laboratories (brand name: Ferrous Ascorbate & Folic Acid Tablets), Glenmark Pharmaceuticals (brand name: Ferrous Ascorbate & Folic Acid Suspension), Alkem Laboratories (brand name: Ferrous Ascorbate & Folic Acid Tablets); Ferrous Fumarate: Pfizer (brand name: Ferrous Fumarate Tablets), Actavis (Teva Pharmaceuticals), Perrigo (brand name: Ferrous Fumarate Tablets); Ferrous Bisglycinate: Albion Minerals, Solgar (brand name: Gentle Iron Bisglycinate), Nature's Bounty (brand name: Iron Bisglycinate); Iron Polymaltose: Abbott Laboratories (brand name: Iron Polymaltose Complex Syrup) Sanofi (brand name: Iron Polymaltose Tablets), Dr. Reddy's Laboratories (brand name: Iron Polymaltose Complex & Folic Acid Tablets); Iron Sucrose: Vifor Pharma (brand name: Venofer), Luitpold Pharmaceuticals (brand name: Iron Sucrose Injection), American Regent (brand name: Iron Sucrose Injection); Iron Dextran: Pharmacosmos (brand name: CosmoFer), American Regent (brand name: DexFerrum), Fresenius Kabi (brand name: INFeD).

As used herein “a scavenger of hemoglobin or heme or a degradation product thereof” refers to a decoy molecule which binds hemoglobin or heme or a degradation product thereof (e.g., bilirubin, biliverdin optionally excess iron depositions) and prevents its penetration to the cell

According to a specific embodiment, the scavenger is selected from the group consisting of hemopexin, heptoglobin and albumin. These are commercially available from various vendors such as listed below. This is a non-exhaustive list. Hemopexin: Sigma-Aldrich (MilliporeSigma), Abcam, R&D Systems (Bio-Techne Corporation); Haptoglobin: MyBioSource, Aviva Systems Biology, Fitzgerald Industries International; Albumin: CSL Behring (brand name: Albuminar), Grifols (brand name: Albutein), Octapharma (brand name: Octalbin).

As used herein “a lysosomal inducer” refers to a molecule which increases the activity of lysosomes, e.g., increases biogenesis, affects pH and/or affects lysosomal integrity.

According to a specific embodiment, the lysosomal inducer is selected from the group consisting of ambroxol, gastrodin, a CDK4/6 inhibitor and an autophagy inducer. It is suggested that interventions to rejuvenate T cell functions act on different modules of the proteostasis network. Those include inducers of autophagy and lysosomal activity, like mTOR inhibitors (Mannick et al., 2014; Mannick et al., 2018), Metformin (Bharath et al., 2020; Yang et al., 2023), and spermidine (Alsaleh et al., 2020), resulting in improved T cell responses and attenuated inflammation.

As used herein “a CD4/6 inhibitor” refers to molecule that act at the Gi-to-S cell cycle checkpoint. This checkpoint is tightly controlled by the D-type cyclins and CDK4 and CDK6.

There a number of CDK4/6 which are already in clinical use, some are listed infra as non-exhaustive examples. Palbociclib (trade name: Ibrance), Ribociclib (trade name: Kisqali), Abemaciclib (trade name: Verzenio), Trilaciclib, Lerociclib, P276-00 and SHR6390

According to a specific embodiment, the d CDK4/6 is selected from the group consisting of Abemaciclib (LY2835219) and PD0332991 (e.g., palbociclib).

Ambroxol is a drug that breaks up phlegm, used in the treatment of respiratory diseases associated with viscid or excessive mucus. A non-exhaustive list of brandnames include: Muciclar, Mucosolvan, Mucobrox, Bisolvon, Cloxan, Mucol, Lasolvan, Mucoangin, Surbronc, Brontex, Ambro, Ambolar, Inhalex, Mucolite (India), Fluibrox and Lysopain.

Gastrodin is a chemical compound which is the glucoside of gastrodigenin. It has been isolated from the orchid Gastrodia elata and from the rhizome of Galeola faberi. It can also be produced by biotransformation of 4-hydroxybenzaldehyde by Datura tatula cell cultures. IUPAC name: 4-(Hydroxymethyl)phenyl β-D-glucopyranoside.

According to a specific embodiment, the autophagy inducer torin1, metformin, resveratrol and spermidine.

Examples of autophagy inducers include, but are not limited to, Rapamycin, CCI-779, Glc, Glc-6-P, Torin1, perhexiline, niclosamide, rottlerin, Lithium, L-690,330, Carbamazepine, sodium valproate, Verapamil, loperamide, amiodarone, nimodipine, nitrendipine, niguldipine, pimozide, Calpastatin, calpeptin, Clonidine, rilmenidine, 2′,5′-Dideoxyadenosine, NF449, Penitrem A, Minoxidil, Penitrem A, SMER10, SMER18, SMER28, SMER analogs, Fluspirilene, trifluoperazine, trehalose, metformin, resveratrol and spermidine.

According to a specific embodiment, the autophagy inducer is selected from the group consisting of torin1, metformin, resveratrol and spermidine.

As used herein “a heme crystal breaking agent” refers to an agent which breaks crystalline forms of heme also typically known as hemozoin (though other forms may exist) in the cell (the structure can be determined by electronic microscopy). According to some embodiments, the heme crystal breaking agent is an anti-malaria drug that interferes with heme aggregation.

The present inventors suggest that heme accumulates in aged T cells as crystals, which makes it less toxic to the cells. Indeed, treating aged T cells with Quinine rescued their proliferative capacity (FIGS. 7A-B). It is suggested that Quinine breaks down Hemozoin crystals, and releases heme that is used by the cells as a source of iron.

According to a specific embodiment, the heme crystal breaking agent is selected from the group consisting of quinine, chloroquine, amodiaquine, quinidine, quinacrine, halofantrine and mefloquine. All these are commercially available such as under the brand names Quinine: Qualaquin, Quinamm, Q-Vel; Chloroquine: Aralen, Avloclor, Resochin; Amodiaquine: Camoquin, Flavoquine; Amodip; Quinidine: Quinidex, Quinaglute, Cardioquin; Quinacrine: Atabrine, Mepacrine; Halofantrine: Halfan; and Mefloquine: Lariam, Mefliam, Mephaquin.

The present teachings are of particular significance in improving the immune response of subjects in need of vaccination.

Thus, according to an aspect of the invention, there is provided a method of vaccinating a subject at least 65 years old, the method comprising administering to the subject a vaccine and an agent which increases the level of labile iron in immune cells and/or reduces toxicity of hemoglobin, heme or a degradation product thereof, thereby vaccinating the subject.

Alternatively, or additionally, there is provided an agent which increases the level of labile iron in immune cells and/or reduces toxicity of hemoglobin, heme or a degradation product thereof as an adjuvant for a vaccine in a vaccination treatment of a subject at least 65 years old.

As used herein “vaccinating” refers to artificial activation of the immune response which occurs through priming the immune system with an immunogen. Stimulating immune responses with an infectious agent is known as immunization. Vaccination includes various ways of administering immunogens. The present teachings relate to both active vaccination and passive vaccination.

According to a specific embodiment, the vaccine is of particular relevance to the elderly population, i.e., those which are at least 65 years old; or having a level of CD4+ T cells which exhibit a level of CD39 expression (per cell) above a predetermined threshold associated with T cell dysfunction (Fang F., Cell Reports 2016, 14 (5): 1218-1231).

The CDC provides guidelines for vaccination per age. According to a specific embodiment, the vaccine is CDC recommended for adults of at least 65 years old

Thus according to some embodiments of the present invention, the vaccine is selected from the group consisting of a flu vaccine (e.g., influenza live attenuated, influenza inactivated and/or influenza recombinant), a pneumococcal vaccine (e.g., PCV15, PCV20, PPSV23), a shingles vaccine, respiratory syncytial virus (RSV) and a tetanus-diphtheria-pertussis vaccine (Tdap).

It will be appreciated that the vaccine can be also against other diseases. For instance, against cancer or autoimmune diseases (e.g., allergies). In such a case the term may be relevant to any agent which promotes innate immunity such as adoptive cell transfer, e.g., CAR-T and more.

As used herein “adjuvant” refers to an immunologic adjuvant which a substance that increases the immune response to a vaccine.

The agent can be administered prior to, concomitantly with or following administration of the vaccine.

According to a specific embodiment, the agent is administered following the vaccine (at least once or more at predetermined intervals).

The agents of some embodiments of the invention can be administered to the subject per se, or in a pharmaceutical composition where it is mixed with suitable carriers or excipients.

As used herein a “pharmaceutical composition” refers to a preparation of one or more of the active ingredients described herein (i.e., agent) with other chemical components such as physiologically suitable carriers and excipients. The purpose of a pharmaceutical composition is to facilitate administration of a compound to an organism.

Herein the term “active ingredient” refers to the agent accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (agents) effective to prevent, alleviate or ameliorate symptoms of a disorder (e.g., a viral disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide blood levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

According to a specific embodiment, two lines of treatments are suggested (1) An acute increase in labile iron to allow T cells to proliferate and activate upon need; and (2) A chronic treatment that will reduce heme load and toxicity in the aged tissues.

(1) An acute need for iron is relevant for boosting vaccination responses and dealing with infections. This can be done by administering labile iron (e.g., ferric ammonium citrate), an iron derivative that is available to the cells and does not require an active uptake through the transferrin receptor or a protein carrier. Alternatively, by administering drugs that can break down heme crystals (e.g., quinine). Heme will then be catabolized by the cells and its bound iron released (i.e., quinine). The administration can be one time or multiple times (e.g., 2-14 times, e.g., 2-6 times).

(2) Chronic administration of heme scavengers, like hemopexin, haptoglobin, serum albumin.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Methods

Mice. Young (7-10 weeks old) and aged (20-23 months old) C57BL/6JOlaHsd female mice were purchased from Envigo (Israel). For aging experiments, retired breeders were purchased at 8 months and housed at the Technion animal facility for additional 12-15 months before used for experiments. C57Bl/6 Rosa26tdTomato/+OT-II mice were received from Prof. Ziv Shulman, The Weizmann Institute of Science. C57 BL/6 CD45.1 OTI mice were generated in Prof. Michael Berger's lab (HUIJ) by crossing C57Bl/6-Tg (TcraTcrb) 1100Mjb/J (OT1; The Jackson Laboratory) with C57Bl/6.SJL PtprcaPep3b; Ly5.1 (CD45.1; The Jackson Laboratory). All mice were housed in specific pathogen-free conditions at The Technion Pre-Clinical Research Authority, and used in accordance with animal care guidelines from the Institutional Animal Care and Use Committee.

T cell isolation and culture. T cells were harvested from mice spleens and/or lymph nodes (inguinal, axillary, brachial, mandibular, and jejunal), and purified by magnetic separation. Depending on the specific experiment, either bulk CD3⁺ T cells or bulk CD3⁺CD4⁺ T cells or pure naïve (CD3⁺CD4⁺CD62L⁺ CD441^{lo} CD25⁻) T cells were purified using commercially available kits (StemCell, 19765A, 19852A, 19851A), following the manufacturer instructions. The protocol for isolating pure naïve T cells from aged mice was modified, as additional antibodies were added to the initial antibody cocktail to overcome changes in T cell population composition in the aged. Specifically, biotinylated anti-mouse/human CD44 (BioLegend, IM7), and biotinylated anti-mouse CD25 (miltenyi 130-123-860, REA568), were added to the company's premade antibody cocktail for negative selection of lymphocytes. Furthermore, the present inventors performed a step of positive selection of CD62L⁺ naïve T cells, using anti-mouse CD62L microbeads (miltenyi, 130-049-701) and magnetic columns (miltenyi, 130-042-201). Primary T cells were cultured at 37° C. and 5% CO₂ in RPMI media, supplemented with: 10% FBS, 10 mM Hepes, penicillin/streptomycin, and 0.035% beta-mercaptoethanol. For activation, cells were cultured on plates pre-coated (overnight at 4° C.) with anti-CD3 (2 μg/mL; clone 145-2C11, BioXCell) and anti-CD28 (4 μg/mL, clone 37.51; BioXCell). Resting cells were supplemented with 5 ng/ml recombinant murine IL-7 (PEPROTECH, 217-17). In some experiments, cell cultures were supplemented with hemin chloride (cayman, 16487), ammonium iron (III) citrate (thermos scientific, A11199.30), BSA (Sigma-Aldrich, A9647), and spleen extract, at different concentrations, as indicated in relevant experiments.

Collection of a interstitial fluid-enriched fraction (referred to as spleen extract; SE). Spleens from young or aged mice were harvested, and gently dissociated ontop of a 70 μM cell strainer, in 5 mL PBS or RPMI, 5 min centrifugation at 1350 RPM to separate the cellular pellet from the fluidic fraction, enriched with interstitial components.

Red blood cell isolation and lysate. Whole blood was collected from dodnor mice via cardiac puncture into MiniCollect EDTA tubes (Greiner, 450531), diluted 1:1 in phosphate-buffered saline (PBS), and subjected to leukoreduction using Hystopaque (Sigma, 10771) at 400 g, without brake, for 30 minutes at room temperature. The luquid phase was carefully removed, and the remaining red blood cell pellets were lysed through 4 freeze/thaw cycles, each lasting 6 min. The resulting lysate was either used immediately or stored at −80° C. until further analysis.

Flow cytometry. For cell-surface staining, cultured T cells or freshly isolated T cells were resuspended in a separation buffer (PBS containing 2% FBS and 2 mM EDTA) and incubated for 20 min, on ice, with an antibody mix, supplemented purified anti-mouse CD16/32 (Biolegend, clone 93). For intracellular staining, True-Nuclear™ Transcription Factor Buffer Set (Biolegend, 424401) was used, following the manufacturer's protocol. Antibodies included in the study: BV510 anti moue CD44 (clone IM7), AF700 anti-mouse CD62 (clone MEL-14), CD3 FITC/BV421 anti-mouse CD3 (clone 17A2) FITC/PE/APC anti-mouse CD4 (clone GK1.5) PerCP anti-mouse CD4 (cloneRM4-5), APC/FITC/BV 421 anti-mouse CD8 (clone 53-6.7), PE/BV 510 anti-mouse CD69 (clone H1.2F3) PE anti-mouse CD39 (clone Duha59), PE/FITC anti-mouse CD25 (clone PC610) CD71 (BV 421, RI7217) all purchased from BioLegend. APC-eFluor 780 anti-mouse CD25 (clone PC61.5, Invitrogen), FITC anti-mouse CD63 (clone NVG-2), AF 647 anti-mouse FOXO1 (W20064D), Recombinant Anti-Ferritin antibody (ab75973), recombinant anti-Heme Oxygenase 1 antibody (ab52947). Cell viability was assessed by Zombie violet or Zombie NIR™ Fixable Viability Kits (423114, 423105, biolegend). Cell proliferation was assessed by cell trace violet proliferation kit (C34557, Invitrogen). Lysosomal activity was determined using a lysosomal intracellular activity assay kit (ab234622) according to the manufacturer's instructions. Intracellular ferrous iron was measured using FerroOrange (Dojindo F374). Cells were incubated with 1 mM of FerroOrange in HBSS at 37° C. for 40 minutes, and analyzed by flow cytometry. For Lysotracker staining, cells were incubated with 62.5 nM of lysotracker deep red (Invitrogen L12492) in HBSS at 37° C. for 40 minutes and analyzed by flow cytometry. All data were collected on the Attune NxT Flow Cytometer (Thermo Fisher) and analyzed using FlowJo (BD).

Functional assays and intracellular ferrous iron staining. Intracellular ferrous iron was measured using FerroOrange (Dojindo, F374). Following extracellular staining, cells were incubated with 1 μM of FerroOrange in HBSS at 37° C. for 30 minutes in a 5% CO2 incubator, and immediately taken for analysis. For Mitochondrial ferrous iron measurement, cells were incubated with 5 μM of Mito-FerroGreen (Dojindo, M489) in HBSS at 37° C. for 30 minutes in a 5% CO2 incubator, washed twice. Lipid peroxidation levels were determined using liperfluo (Dojindo, L248) according to the manufacturer's instructions or with BODIPY™ 581/591 C11 (D3861, Invitrogen) at a concentration of 5 M in PBS at 37° C. for 30 minutes in a 5% CO2 incubator. Intracellular ROS was measured using by DCFDA-Cellular ROS Assay Kit (AB113851) according to the manufacturer's instructions. All assays were analyzed by flow cytometry.

Untargeted, whole-cell Proteomics. T cells were isolated from young and aged mice spleens, and sorted on the by flow cytometry (BD FACS Aria Illu) to yield pure naïve T cells (CD4⁺CD62L^{hi}CD44^{lo}CD25⁻). Cell pellets were frozen immediately following sorting or after 24 hr of activation. Total cell proteins were extracted, digested by trypsin, and analyzed by LC-MS/MS on Q-Exactive plus (ThermoFisher). 2 μg protein/sample were loaded. Collected data were processed using Maxquant (Mathias Mann, Max Planck Institute) and identified against the mouse proteome (Uniprot database (January 2020)), and a decoy database.

Adoptive T cell transfer. Young T cells were isolated from spleens of young OTII td-tomato transgenic mice (8-12 weeks-old) by magnetic isolation (StemCell), as described above. 5×10⁶ cells in 200 ul of PBS were transferred by tail vein injection into wild type C57Bl/6 young or aged recipients. Two weeks following cell transfer, mice were euthanized and spleen and lymph nodes (inguinal, axillary, brachial, mandibular, and jejunal) were extracted for further analysis.

Heme/bilirubin quantitation. 50 mL of spleen extract were mixed with 200 mL of heme reagent (Heme Assay Kit, Sigma-Aldrich, MAK316), or with 200 mL of total/direct bilirubin reagent from (Bilirubin Assay Kit, Sigma-Aldrich, MAK126) and incubated for 5-10 min. Absorption was measured at 400 nm (heme) or 530 nm (bilirubin) on a plate reader (Biotek). Reads were normalized to tissue weight or to the total cell number. For intracellular heme content, 5×10⁶ cells were lysed in 60 mL RIPA buffer (Sigma-Aldrich, R0278) containing a protease inhibitor cocktail (1:500, Abcam, ab201111). Cell lysates were incubated for 15 min on ice, followed by 3 cycles of 15 min incubation at −80° C. and 10 min incubation at room temp, with vortex. Lysates were then centrifuged for 15 min at 11,000 RPM at 4° C. 50 mL of cell lysates were mixed with 200 mL of heme reagent (Heme Assay Kit, Sigma-Aldrich, MAK316), and absorption was measured.

Fluorescent microscopy. Cells were stained simultaneously with lysotracker deep red and FerroOrange in HBSS as described above, and imaged using Leica DMI8 inverted fluorescent microscope.

Histology. Spleens harvested from young and aged mice were fixed with 4% PFA and processed in Tissue Processor (Leica TP1020, Germany). Processed tissue was paraplast paraffin-embedded and 4 mM sequential sections were made (Leica RM2265 Rotary Microtome, Germany). Sections were stretched on a warm 37° C. water bath, collected into slides, and dried at 37° C. overnight. Sections were deparaffinized in xylene and rehydrated and processed for H&E and Perls Prussian blue staining. Stained slides were scanned by 3DHistech Pannoramic 250 Flash III and visualized using the Case Viewer software (3DHISTECH).

Immunohistochemistry. Tissue dissection, fixation, embedding and sectioning was performed as previously described (STAR Protoc 2021 Jun. 18; 2 (3): 100499). For staining, slides were wased (PBS), permeabilized (0.1% Triton) and blocked (5% BSA), followed by an overnight 4° C. incubation with AF647 anti-CD169 (in 1% BSA). Slides were then washed (PBS) and incubated with a secondary antibody for 1 hr at RT, followed by a nuclear staining (DAPI, 0.5 μg/ml). Finally, slides were washed (PBS), dried, and mounted (Mounting Fluorogel Medium) before storage at prior to imaging using an LSM710 AxioObserver microscope.

Quantitative, real-time PCR (qPCR). Total RNA was extracted and from CD3⁺ T cells, using QuickRNA Micro prep Kit (ZYMO RESEARCH R10200). cDNA was synthesized using the High-Capacity cDNA RT kit (Applied Biosystem, 4374966). Quantitative PCR was run on QuantStudio™ 3 Real-Time PCR System, 96-well (Applied Biosystem), using Fast SYBR Green Master Mix (Applied Biosystems, 4385612). Primers used: Blvra: F: 5′-AAGATCCCGAACCTCTCTCT-3′ (SEQ ID NO: 1), R: 5′-TTATCAAGGCTCCCAAGTTCTC 3′ (SEQ ID NO: 2); Blvrb: F: 5′-AAGCTGTCATCGTGCTACTG-3′ (SEQ ID NO: 3), R: 5′ CAGTTAGTGGTTGGTCTCCTATG-3′ (SEQ ID NO: 4); Fth1: F: 5′-TCAACCGCCAGATCAACC-3′, (SEQ ID NO: 5); R: 5′-TCAGTTTCTCGGCATGCTC-3′ (SEQ ID NO: 6); Ftl1: 5′-CGTGGATCTGTGTCTTGCTTCA-3′ (SEQ ID NO: 7), R: 5′-GCGAAGAGACGGTGCAGACT-3′ (SEQ ID NO: 8); Hmox1: F: 5′-GTTCAAACAGCTCTATCGTGC-3′ (SEQ ID NO: 9), R: 5′-TCTTTGTGTTCCTCTGTCAGC-3′ (SEQ ID NO: 10); Rps18: F: 5′ 5′-CCGCCATGTCTCTAGTGATCC-3′ (SEQ ID NO: 11), R: GGTGAGGTCGATGTCTGCTT-3′ (SEQ ID NO: 12).

Vaccination and in vivo iron supplementation. Young T cells were isolated from young OTII-td-tomato or OTI-CD45.1 transgenic mice by magnetic separation, and equally mixed at a 1:1 ration. A total of 4×10⁶ cells (OTI and OTII) were inoculated by tail vein injection into wild-type C57Bl/6 young or aged recipients. 3 weeks follow cell transfer, recipient mice were injected intraperitoneally (i.p.) with OVA albumin (sigma, A7642) adsorbed in 40% Alum adjuvant (Serva: 12261) or with 0.9% saline. On days 1 and 4 following vaccination, some mice received intravenous (i.v.) injections of ferric ammonium citrate (FAC; 900 μg). Mice were sacrificed on day 5 following vaccination, the spleen was harvested and CD3+ T cells were isolated using a magnetic isolation kit (StemCell). Samples were analyzed by flow cytometry to quantify percentages of OTII td-tomato+ and OTI-CD45.1+ positive T cells.

### Example 1

### The Aged Spleen Microenvironment Induces T Cell Dysfunction

Generation of naïve T cells is entirely dependent on the thymus, which undergoes involution during childhood and adolescence. Thus, in humans, the naïve T cell pool is maintained throughout life mainly by low-grade proliferation of existing naïve T cells. In old age, reduced rate of naïve T cell proliferation and accelerated loss of cellular quiescence (priming/differentiation) account for a shift in the phenotype of the T cell population⁷. Importantly, priming, and increased differentiation of naïve T cells in old age are seen also in mice kept under pathogen-free conditions, suggesting that aging of the host itself could promote T cell aging independent of immunological history⁸.

Naïve T cells reside in secondary lymphoid tissues, the spleen and lymph nodes. To investigate the potential effect of the tissue microenvironment on resident T cells, naïve CD4⁺ T cells (CD4⁺CD25⁻CD62L⁺CD441^{lo}) were purified from the spleen and lymph nodes of aged (21-23 months old) C57Bl/6 mice and analyzed their response to ex vivo activation (FIG. 1A). Surprisingly, major functional differences were found between naïve T cells derived from the spleen and lymph nodes of the same mouse. Specifically, T cells derived from the aged spleen were less viable (FIG. 1B), expressed lower levels of CD69, an early activation marker (FIG. 1C), and showed lower proliferative capacity than cells derived from aged lymph nodes (FIG. 1D). A recent paper identified CD39, as a prominent marker of T cell dysfunction in aging and exhaustion^{9,10}. The present results verified CD39 elevation in aged T cells compared to young T cells. Interestingly, CD39 overexpression was more pronounced in T cells derived from the aged spleen (FIG. 1E). The naïve T cell compartment shrinks with aging. It was found that this shift in population composition was more pronounced among CD4+ T cells residing in the aged spleen, compared to aged lymph nodes (FIG. 1F). Lymphoid organs were collected from mice at the ages of 2, 12 and 20 months, and analyzed by flow cytometry. The present inventors gated on CD4⁺CD25⁻ T cells and characterized the populations based on expression of CD62L and CD44, as shown in FIG. 1F, to quantify naïve, central memory (CM) and effector memory (EM) T cells. To directly examine whether exposure to the aged spleen microenvironment, in vivo, was sufficient to induce functional defects, CD4⁺ T cells were isolated from young td-Tomato transgenic mice. T cells derived from these mice constitutively express the fluorescent protein td-Tomato. Td-Tomato⁺ T cells were transfused intravenously into young or aged recipients. After two weeks, recipient mice were sacrificed, and T cells harvested from their spleens and lymph nodes were stimulated ex vivo to assess proliferation (FIG. 1G). Strikingly, proliferation of tdTomato+ T cells derived from aged spleens was significantly lower compared to T cells purified from the LN of the same mice, or from young hosts (FIG. 1H). Young T cells residing in aged spleens showed a significant increase in cell size (FIG. 1I), and elevated CD39 levels (FIG. 1J). Differences in CD39 expression maintained after activation (not shown). Together, these results show that the microenvironment within the aged spleen promotes T cell dysfunction.

### Example 2

T Cells in the Aged Spleen are Enriched with Proteins Associated with Stress and Inflammation

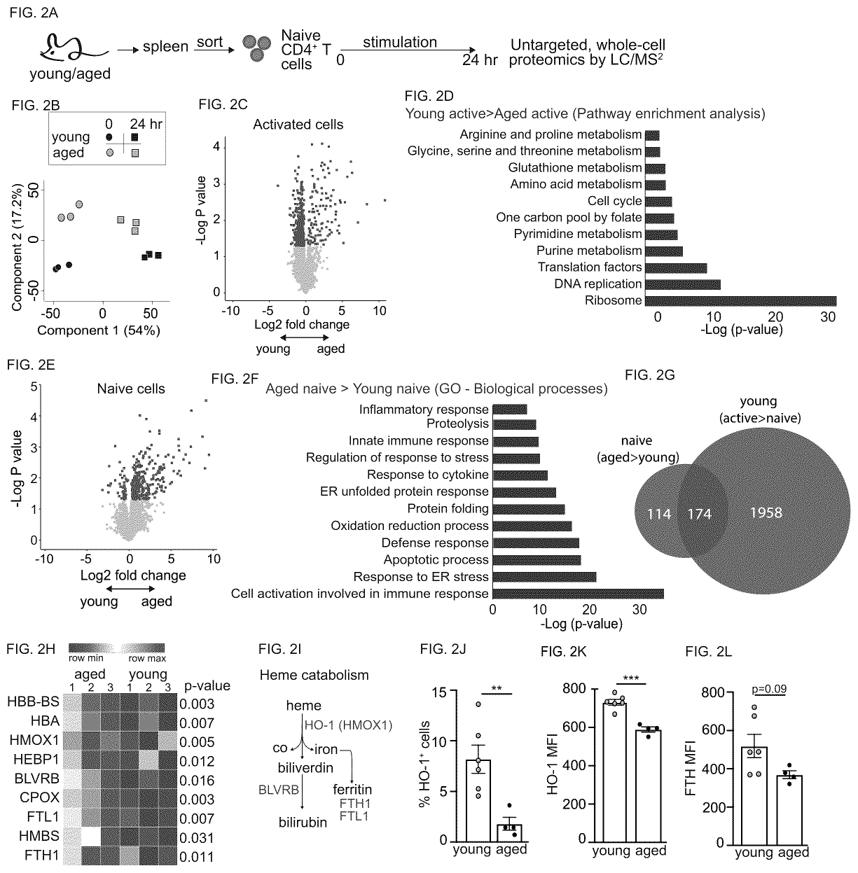

To identify in T cells the cellular response to the aged spleen microenvironment, whole-cell, label-free proteomics analysis was performed of pure naive CD4⁺ T cells collected from the spleens of young and aged mice. Cells were immediately processed or stimulated ex vivo for 24 hr prior to protein extraction, peptide degradation and analysis by LC-MS/MS (FIG. 2A). The dynamic changes of over 3800 proteins were determined. According to principal component analysis (PCA), the largest changes in protein composition were induced by activation (PCA1 represents 54% of the variance and separates between 0 and 24 hr). Age accounted for 17.2% of the variance (PCA2, separating young and aged T cells; FIG. 2B). 692 proteins were differentially expressed between young and aged T cells post-activation. 84% of those were higher in young T cells (FIG. 2C). Pathway enrichment analysis highlighted multiple metabolic pathways that were significantly over-represented in young vs aged T cells. These included: one-carbon metabolism, purine and pyrimidine metabolism, amino acids metabolism and the urea cycle. Enrichment of proteins involved in DNA replication and protein translation was identified (FIG. 2D). Despite their naïve identity and the strict sorting parameters of pure naïve T cells, a large number of proteins (330) were differentially expressed between naïve young and aged T cells (FIG. 2E). 87% of them were overrepresented in aged T cells and were enriched with proteins associated with activation, proinflammation, and stress responses (FIG. 2F). A majority of these proteins is normally induced by activation in young T cells (FIG. 2G). To identify specific pathways induced in aged naive T cells compared to young, the present inventors performed pathway enrichment analysis of the top 100 proteins. This analysis highlighted proteins associated with heme metabolism and degradation (FIG. 2H). Heme is catabolized by heme oxygenase 1 (HMOX1 or HO-1) to generate CO, biliverdin, and labile iron. Excess iron is stored in cells in complex with ferritin, a globular protein composed of 24 subunits of two types: FTH and FTL. Biliverdin is further reduced to bilirubin by BLVRB (FIG. 2I). All these proteins were significantly overexpressed in aged naïve T cells compared to young. Overexpression of HO-1 (FIGS. 2J, 2K) and ferritin (FIG. 2L) in naïve CD4⁺ T cells from aged mice compared to young were further verified by flow cytometry.

### Example 3

### Heme and the Products of its Degradation are Accumulating in Aged Spleens

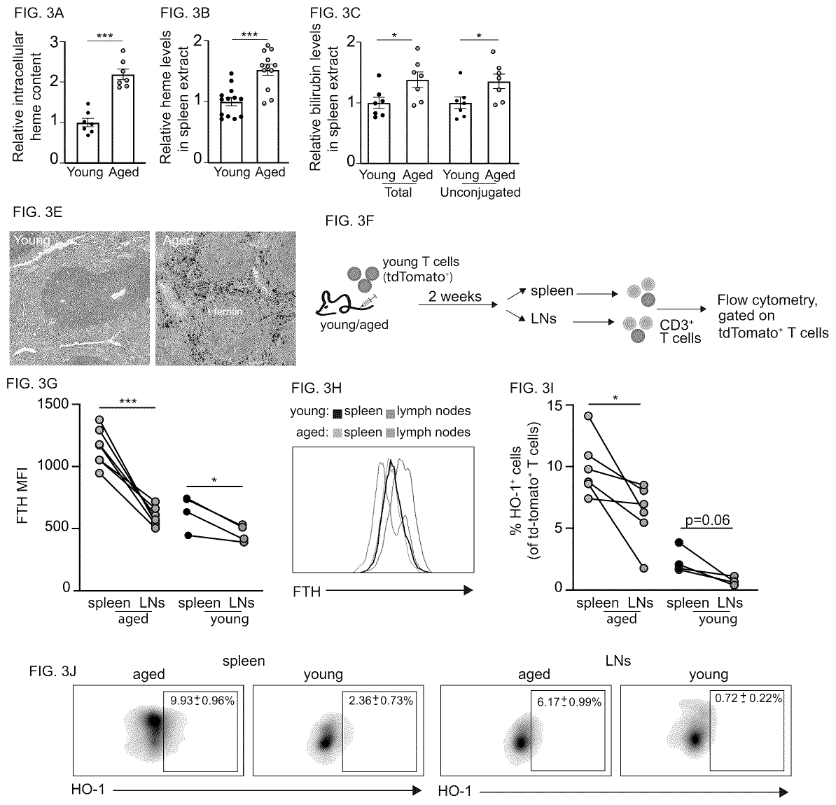

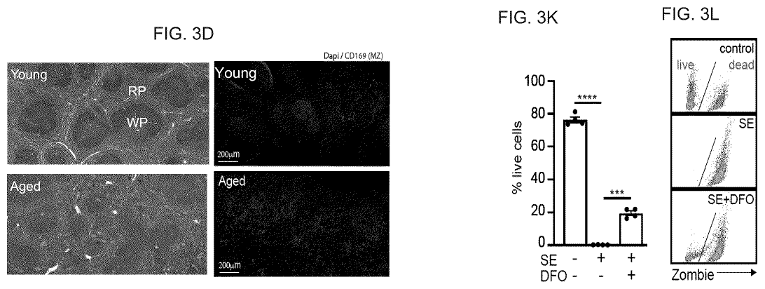

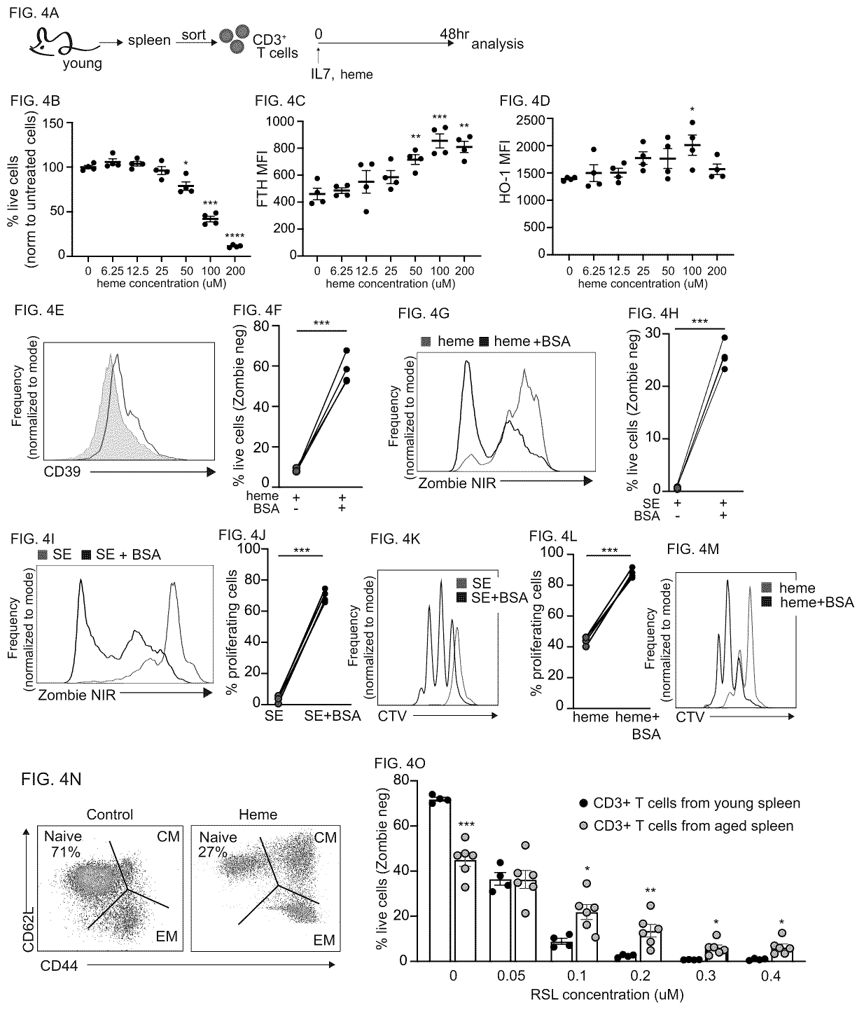

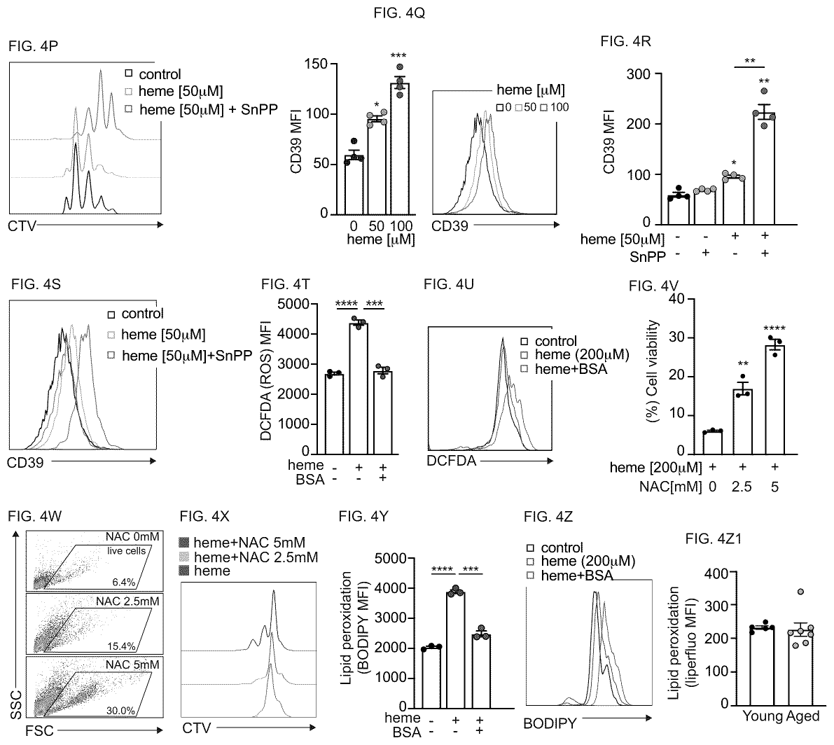

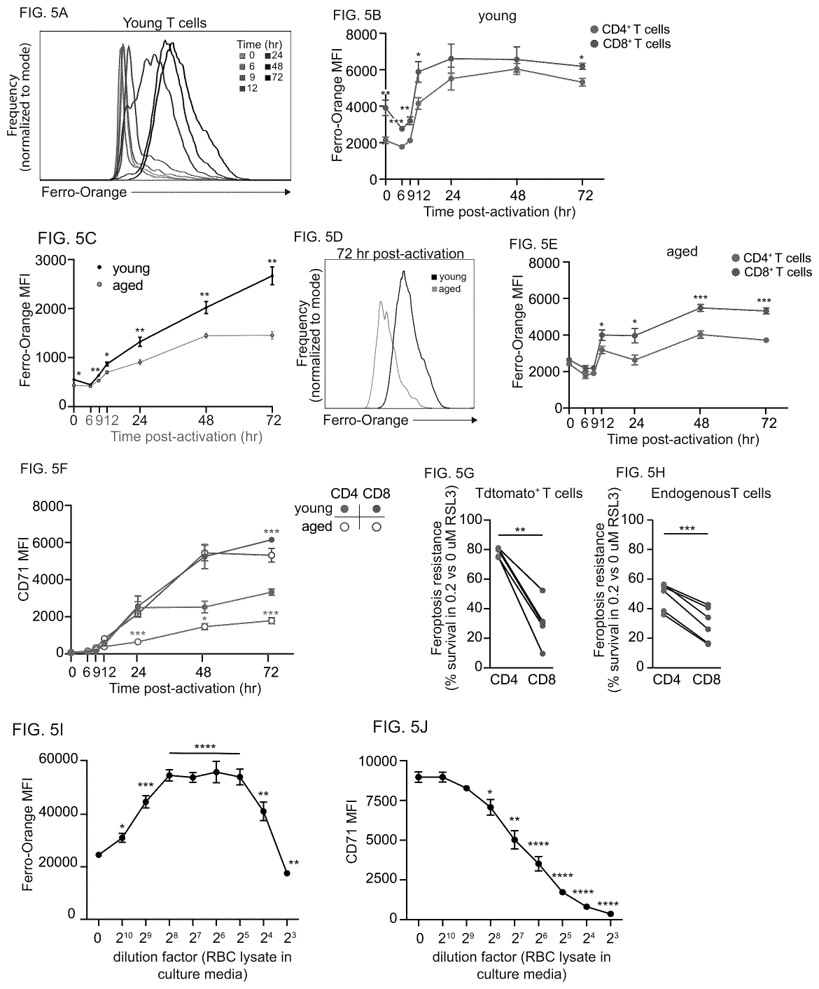

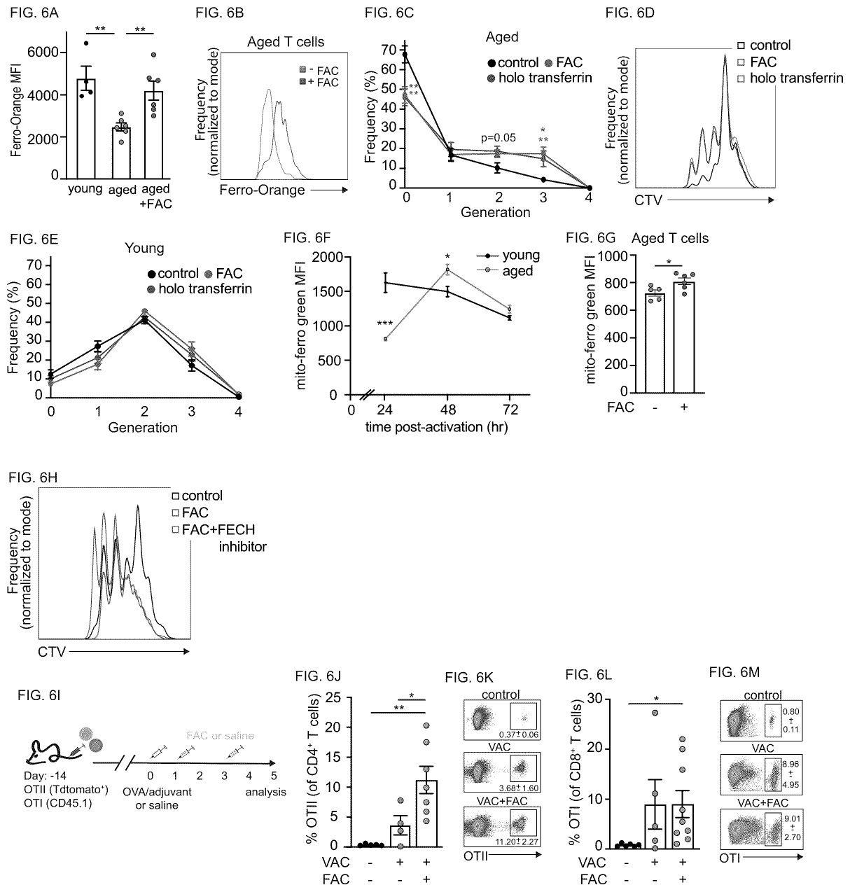

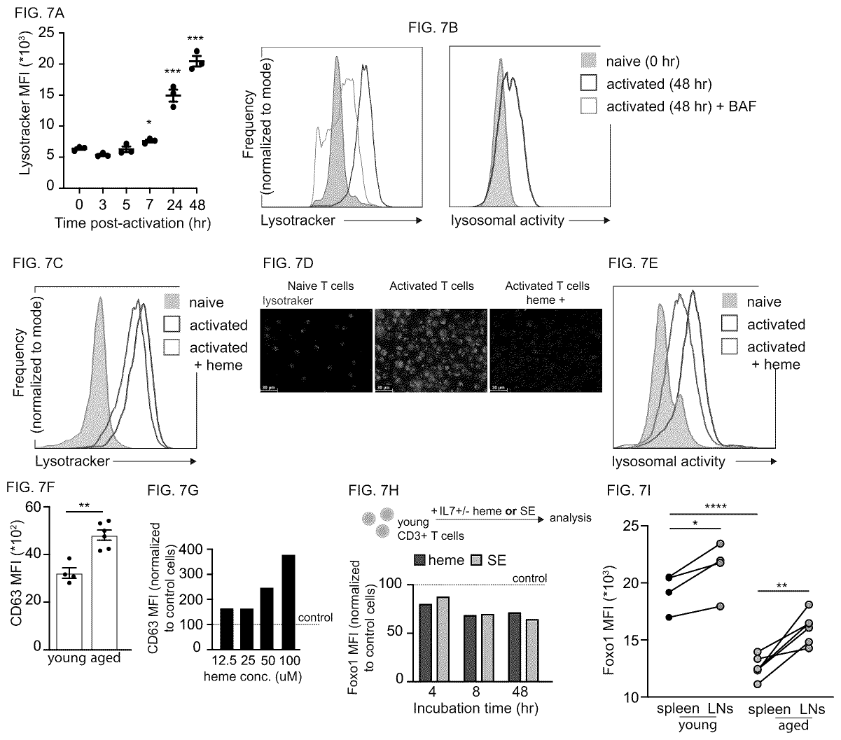

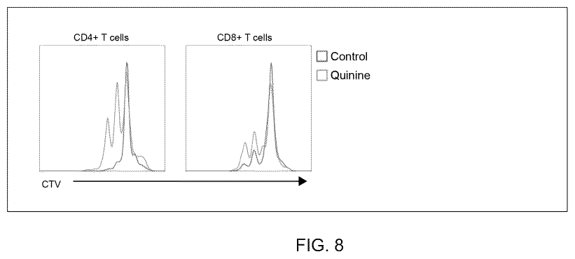

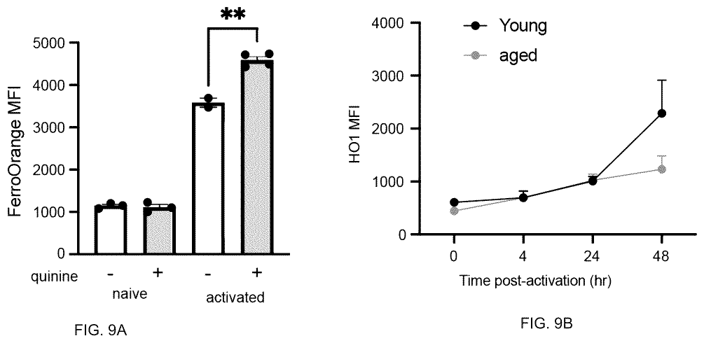

Heme is an interesting target when looking for a deleterious signal that is specifically affecting T cells in the aged spleen and not LNs, as it is directly connected to the spleen being a site of senescent red blood cells (RBCs) removal and iron recycling by red pulp macrophages. To examine whether aged T cells were indeed exposed to high heme concentrations in vivo, colorimetric assays were used to quantify heme levels in spleen extracts and intracellularly, in T cells isolated from the spleen of young and aged mice. Indeed, elevated levels of heme were detected in CD3⁺ T cells isolated from aged spleens (FIG. 3A), and in aged spleens extracts, compared to young (FIG. 3B). In agreement, higher levels of secreted bilirubin were found in aged spleen extracts (FIG. 3C). The spleen is organized in regions called the red pulp (RP) and white pulp (WP), separated by an interface called the marginal zone. The red pulp serves mostly to filter blood and recycle iron from senescent red blood cells. The white pulp is similar in structure to a lymph node, containing T- and B-cell zones. With aging, the spleen infrastructure is compromised, as the borders between the red pulp and white pulp become less clear (FIG. 3D, and ¹¹). The present H&E staining further highlighted increased brown depositions in aged spleen tissues. Previous studies identified these to be iron-reach aggregates called hemosiderin, which is indicative of high iron load in the aged tissue (FIG. 3D; and ¹²). Additionally, the Prussian Blue method was used to detect iron depositions in the spleen. As previously reported, the signal was higher in aged tissue, and could also be detected inside white pulp areas. Perl's Prussian Blue detects iron in the ferric state, mainly iron bound to ferritin or its aggregated form, hemosiderin (FIG. 3E; and ¹²). FIG. 3D right panel shows representative images of frozen spleen sections stained with anti-CD169 to mark marginal zone (MZ) macrophages. To directly test whether exposure to an aged spleen microenvironment was sufficient to induce the specific protein signature associated with T cells from aged spleens, young T cells derived from tdTomato transgenic mice were transferred into young and aged recipients. After two weeks, the mice were sacrificed and CD3⁺T cells were purified and analyzed by flow cytometry, gating on tdTomato⁺ T cells. Strikingly, young T cells residing in the aged spleen but not lymph nodes significantly upregulated ferritin (FIGS. 3D, 3H) and HO-1 (FIGS. 31, 3J). FIG. 3K and FIG. 3L show the detrimental effect of aged spleen extract on T cell viability. Addition of DFO an iron chelator, partially rescued cell viability.

### Example 4

### Heme and Spleen Extract (SE) Drive Aging-Like Phenotypes in Young T Cells

To directly examine whether heme itself could cause T cell dysfunction, CD3⁺ T cells were purified from the spleens of young mice and cultured ex vivo in the presence of IL-7, to maintain a resting, naïve phenotype (FIG. 4A). Exposure to heme significantly reduced cell viability, in a dose-dependent manner (FIG. 4B) and was sufficient to induce expression of ferritin and HO-1 in young T cells (FIGS. 4C and 4D, respectively), as seen in young T cells residing in an aged spleen (FIGS. 3F-3J). CD39, a marker of dysfunctional aged T cells was also induced on young T cells exposed to heme (FIG. 4E). Thus, exposure to heme was sufficient to drive multiple phenotypes that resemble aged T cells.

Albumin is the most abundant heme binding protein in the serum. Interestingly, serum levels of albumin and other heme binding proteins (hemopexin, haptoglobin) are significantly reduced with aging¹³. The present inventors examined whether serum albumin (BSA) could rescue T cells exposed to heme or spleen extract (SE). To this end, young T cells were activated for 48 hr using plate bound anti-CD3/anti-CD28, in the presence of heme or SE, with and without BSA. Indeed, BSA increased viability of T cells activated in the presence of heme (FIGS. 4F, 4G) or SE (FIGS. 4H, 4I). Similarly, proliferation was completely blocked by exposure to SE or heme and was rescued with BSA (FIGS. 4J-4K, and 4L-4M, respectively). Next, the present inventors tested whether exposure to heme contributed to the observed differences in the composition of T cell populations between spleen and lymph nodes of aged mice (FIG. 1F). T cells were isolated from the spleen of young mice and incubated in the presence of heme, without TCR-mediated stimulation. Strikingly, exposure to heme was sufficient to induce T cell differentiation and deplete the naïve T cell pool. (FIG. 4N). These results suggest that heme is sufficient to drive aging phenotypes in young T cells. Sequestering heme from the microenvironment protects T cells from these deleterious effects. The old splenic tissue is loaded with protein aggregates containing heme and iron (FIGS. 3D and 3E and ¹²). The present inventors hypothesized that the unique protein signature measured in aged T cells (FIG. 2H) allows them to survive this hostile microenvironment. To directly test this hypothesis, CD3⁺ T cells derived from the spleen of young and aged mice were cultured ex vivo with increasing doses of RSL, a potent inducer of ferroptosis (iron-induced cell death). While young control T cells were significantly more viable than aged T cells, treatment with RSL reversed this phenotype as aged T cells survived even high levels of the drug in significantly higher concentrations (FIG. 4O).

T cells treated with heme together with SnPP showed proliferation arrest even when treated with lower concentrations of heme (50 μM; FIG. 4P). In agreement with the present finding that the aged spleen microenvironment induced CD39 expression on T cells (FIGS. 1A-J), it was found that exposure to heme itself was sufficient to induce CD39 on young T cells (FIG. 4Q). Like proliferation arrest, CD39 expression was directly induced by heme, as addition of SnPP caused an even greater boost in CD39 levels, even under lower concentrations of heme (FIGS. 4R-S). Thus, exposure of young T cells to heme drove multiple phenotypes characteristic of aged T cells.

Heme is a potent inducer of reactive oxygen species (ROS) (Voltarelli et al., 2023). Indeed, T cells exposed to heme showed an approximate 2-fold increase in cellular ROS and its complete abolishment by BSA (FIGS. 4T and 4U). Addition of the antioxidant N-acetyl cysteine (NAC) improved cell viability (FIGS. 4V, 4W) and proliferation (FIG. 4X) of young T cells exposed to heme, suggesting that heme induced aging phenotypes in young T cells was partly mediated by ROS. Similarly, NAC improved cell viability (not shown) and proliferation (not shown) of T cells cultured with spleen extract. ROS could promote lipid peroxidation that will eventually result in cell death by ferroptosis (Yang and Stockwell, 2016). Indeed, a large induction in lipid peroxidation was measured in T cells exposed to heme, and was rescued by BSA (FIGS. 4Y, 4Z). Taken together, these results suggest that heme accumulation in the aged spleen microenvironment induces oxidative stress in T cells and could lead to cell death by ferroptosis. However, neither BSA nor NAC improved aged T cells survival and proliferation (not shown). Moreover, quantification of lipid peroxidation in aged T cells compared to young showed no apparent difference (FIG. 4Z1). Thus, components accumulating in the aged spleen microenvironment and specifically heme, induce ROS, lipid peroxidation, cell death and proliferation arrest in young T cells. Yet, aged T cells residing in the aged spleen seem to be resistant to lipid peroxidation and they persist in vivo. Thus, it is hypothesized that aged T cells developed mechanisms that allowed them to survive the hostile microenvironment of the aged spleen.

### Example 5

### Aged T Cells are Iron Deficient

The present inventors hypothesized that ferroptosis resistance was accomplished by maintaining low cellular iron levels. To test this, FerroOrange was employed, a fluorescent probe that interacts with cellular ferrous ion (Fe⁺²), to measure changes in labile iron in T cells following activation. In young T cells, labile iron pools increased as early as 9 hr post-activation and continued rising until 72 hrs (FIG. 5A). Iron levels varied across different populations as Ferro-Orange fluorescence intensity was higher in CD8⁺ compared to CD4⁺ T cells, pre- and post-activation (FIG. 5B). In agreement with the present hypothesis, labile iron levels were significantly lower in aged T cells compared to young (FIGS. 5C, 5D). The differences in iron content between CD4⁺ CD8⁺ T cells increased with age (FIGS. 5E vs. 5B), suggesting that CD4⁺ T cells were more susceptible to age-related iron deficiency. A major pathway for increasing cellular iron is the uptake of transferrin-bound iron, mediated by the transferrin receptor (TFR1), also known as CD71. Young T cells upregulated CD71 immediately upon activation (not shown and (Motamedi et al., 2015)). In agreement with the observed differences in labile iron, CD8⁺ T cells expressed higher levels of CD71 compared to CD4⁺ T cells (FIG. 5F). With aging, only CD4⁺ T cells showed a significant drop in CD71 (FIG. 5F). To further connect low cellular iron to ferroptosis resistance, the present inventors reanalyzed the adoptive transfer experiment (injecting young TdTomato+ T cells into young and aged hosts) shown in FIG. 5G, to assess ferroptosis resistance in specific T cell populations derived from the aged spleen. Strikingly, CD4⁺ T cells were more resistant to RSL3-induced ferroptosis compared to CD8⁺ T cells. This was seen in both TdTomato⁺ T cells (young T cells residing in the aged spleen; FIG. 5G) and TdTomato− T cells (aged endogenous T cells; FIG. 5H). Together, these data showed the correlation between ferroptosis resistance and low cellular iron levels, and suggested that this was mediated, at least in part by downregulating CD71. To further establish the causal relation between ferroptosis resistance, iron uptake and cellular labile iron pools, young T cells were activated in media supplemented with increasing concentrations of RBC lysate. Activation induces iron uptake, and cells cultured in low levels of RBC lysate increased cellular iron pools even more, in agreement with RBC lysate being a rich source of iron. However, this trend flipped with increasing amounts of RBC lysate added (FIG. 5I), together with a prominent downregulation of CD71 (FIG. 5J).

### Example 6

### Iron Supplementation Rescued Vaccination Responses in Aged T Cells

The present findings highlight depletion of labile iron pools as a mechanism to resist ferroptosis in the aged spleen microenvironment. It is thus hypothesized that iron deficiency impairs proliferation in these cells (as demonstrated in FIGS. 1A-J). To examine this hypothesis, the present inventors first tested whether labile iron pools in aged T cells could be replenished by supplementing them with ferric ammonium citrate (FAC), an iron derivative that bypasses transferrin receptor. Aged T cells were activated ex-vivo in the presence of FAC. Analysis of cellular iron levels using FerroOrange verified that FAC increased labile iron pools in treated aged T cells (FIG. 6A, 6B). An additional indication of increased labile iron content was the downregulation of CD71 on FAC-supplemented T cells. In CD8⁺ T cells, the shift in CD71 expression was smaller, in agreement with above results showing that iron deficiency is more prominent in aged CD4⁺ T cells (not shown).

To examine whether iron supplementation could rescue proliferation in aged T cells, cells were stimulated ex vivo in the presence of FAC or holo-transferrin (iron saturated transferrin). Both treatments led to a significant improvement in proliferative capacity compared to non-treated aged T cells (FIGS. 6C, 6D). The fact that holo transferrin supplementation rescued proliferation in aged T cells suggested that the mechanism of iron uptake through CD71 was intact in these cells. In young T cells, iron supplementation did not affect proliferation (FIG. 6E), suggesting that iron was not a limiting factor in these cells. Young T cells ‘parking’ in an aged spleen for 2-3 weeks showed ferroptosis resistance and impaired proliferation. To test whether those transferred cells were iron deficient, the present inventors transferred young TdTomato⁺ T cells into aged or young recipients. 2-3 weeks following the transfer, the mice were sacrificed, and T cells stimulated ex vivo with and without iron supplementation (not shown). Iron supplement did not affect proliferation of T cells residing in young spleens, but improved proliferation of T cells isolated from the aged spleen. Iron is essential for multiple fundamental cellular processes required for proliferation, including DNA synthesis, and mitochondrial biogenesis during early activation (Ron-Harel et al., 2016; Yarosz et al., 2020). Previous studies highlighted defects in mitochondrial biogenesis and nucleotide biosynthesis in aged T cells following activation (Ron-Harel et al., 2018). To directly measure mitochondrial labile iron pools in young and aged T cells, the present inventors used mito-Ferro green (Nishizawa et al., 2020). Mitochondrial iron was significantly lower in aged T cells at 24 hours post-activation, a critical time for mitochondrial biogenesis (Ron-Harel et al., 2016). FAC supplementation replenished mitochondrial iron pools (FIG. 6F). In the mitochondria, iron is incorporated into protoporphyrin in the last step of heme biosynthesis, in an enzymatic reaction mediated by ferrochelatase (FECH). Addition of a FECH inhibitor (N-methyl protoporphyrin IX) to aged T cells supplemented with FAC prevented the alleviating effect on proliferation (FIG. 6H). Together, these data show that iron deficiency inhibits aged T cells proliferation and activation, in part by inhibiting mitochondrial activity and metabolism.

The connection between iron availability and T cells response to vaccination was demonstrated in a recent study that used hepcidin to induce iron deficiency in mice. T cells response to vaccination was diminished in mice treated with hepcidin and rescued by in vivo administration of FAC (Frost et al., 2021). In this study, the present inventors discovered that T cells, and specifically CD4⁺ T cells, developed iron deficiency to survive the in vivo milieu of the aged spleen, and took a similar approach to test whether iron supplementation could improve T cells response to vaccination in aged hosts. T cells with a known antigen specificity against ovalbumin (OVA) were isolated from transgenic young mice and injected into aged recipients. Each mouse received a mixture of OVA-specific OTI (CD8⁺CD45.1⁺) and OTII (CD4⁺TdTomato⁺) T cells. 3 weeks following cell transfer, recipient mice were vaccinated intraperitoneally with OVA emulsified in Alum adjuvant. Control mice were injected with saline. FAC was administered to vaccinated mice intravenously on days 1 and 3 following vaccination. Control mice received saline. Mice were sacrificed on day 5, and T cell content in the spleen was analyzed by flow cytometry (FIG. 6I). A timed iron supplementation post vaccination improved proliferation of antigen specific CD4⁺ T cells (OTII; FIG. 6J, 6K). The proliferation of antigen specific CD8⁺ T cells was not affected by FAC administration (FIGS. 6L, 6M), in agreement with our data showing that these cells did not suffer iron deficiency (FIG. 5A-J).

### Example 7

### Heme Induces Iron Deficiency by Inhibiting Lysosome Activity

The major pathways for increasing labile iron are (1) uptake of transferrin-bound iron (endocytosis) and (2) iron release from intracellular ferritin stores (ferritinophagy;¹⁶. Both pathways require active lysosomes to yield labile iron. As demonstrated in previous studies, T cell activation induced lysosomal biogenesis (quantified by lysotracker; FIG. 7A) and increased lysosomal activity (FIG. 7B). Strikingly, exposure to heme, downregulated lysosomal mass, quantified by flow cytometry (FIG. 7C) and fluorescent microscopy (FIG. 7D). Also lysosomal activity was suppressed in T cells activated in the presence of heme (FIG. 7E). Lysosomal dysfunction results in accumulation of late endosomes, also referred to as multi vesicular bodies (MVBs), that are identified by expression of CD63. This was specifically demonstrated to be the case in aged T cells (FIG. 7F; ¹⁷). In agreement, it was found that treatment with heme induced CD63 expression, in a dose-dependent manner (FIG. 7G). FOXO1 regulates expression of TFEB, the master regulator of lysosomal biogenesis¹⁷. Moreover, FOXO1 expression is critical for maintaining T cell quiescence, and its deficiency induces T cell senescence¹⁸. The present inventors hypothesized that the microenvironment in the aged spleen suppressed FOXO1 expression, thus inducing T cell priming (FIG. 7E-G) and dysregulates lysosomal biogenesis and activity. To test this hypothesis, FOXO1 levels were quantified in resting young T cells that were incubated ex vivo with either heme or SE. Strikingly, FOXO1 levels dropped within 4 hr and continued to drop during the 48 hr of incubation (FIG. 7H). Finally, the present inventors measured FOXO1 expression in T cells isolated from the spleen and LNs of young and aged mice and found that FOXO1 expression in T cells, in vivo, depended on the microenvironment. Specifically, FOXO1 expression was significantly lower in T cells residing in the aged spleen compared to T cells in the aged LNs (FIG. 7I).

### Example 8

Quinine Releases Iron from Intracellular Heme Pools

Hemolytic parasites are avoiding heme toxicity by crystalizing excess heme into large crystals called Hemozoin. Approved anti-malaria drugs, like Quinine, are breaking down these crystals thereby killing the parasites. The fact that these crystals can form also in mammalian cells has only recently been demonstrated, by a study that showed Hemozoin accumulation in genetically manipulated macrophages (Pek et al. eLife. 2019; 8: e49503.).

The above results show that heme accumulates in aged T cells, therefore it was hypothesized that it is accumulating in crystals, which makes it less toxic to the cells. In support of this thesis, it was found that treating aged T cells with Quinine rescued their proliferative capacity (FIG. 8). It is suggested that Quinine broke down Hemozoin crystals, and released heme that was used by the cells as a source of iron.

FIGS. 9A-B shows that quinine increases labile iron pools in aged T cells, as determined by iron measurement (FIG. 9A) and HO-1 levels (FIG. 9B). This data demostrates that quinine could release intracellular iron stores in aged T cells. It is suggested that this is the result of the release of heme from hemosoin and its catabolism by HO-1, which expression increases following activation.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Included in the Document

ADDIN EN.REFLIST1 Yousefzadeh, M. J. et al. An aged immune system drives senescence a ageing of solid organs. Nature 594, 100-105, doi:10.1038/s41586-021-03547-7 (2021).
2 Desdin-Mico, G. et al. T cells with dysfunctional mitochondria induce multimorbidity and premature senescence. Science 368, 1371-1376, doi:10.1126/science.aax0860 (2020).
3 Franceschi, C. et al. Inflamm-aging. An evolutionary perspective on immunosenescence. Ann NY Acad Sci 908, 244-254 (2000).
4 Ferrucci, L. & Fabbri, E. Inflammageing: chronic inflammation in ageing, cardiovascular disease, and frailty. Nat Rev Cardiol 15, 505-522, doi:10.1038/s41569-018-0064-2 (2018).
5 Mittelbrunn, M. & Kroemer, G. Hallmarks of T cell aging. Nat Immunol 22, 687-698, doi:10.1038/s41590-021-00927-z (2021).
6 Goronzy, J. J. & Weyand, C. M. Successful and Maladaptive T Cell Aging. Immunity 46, 364-378, doi:10.1016/j.immuni.2017.03.010 (2017).
7 Goronzy, J. J. & Weyand, C. M. Mechanisms underlying T cell ageing. Nat Rev Immunol 19, 573-583, doi:10.1038/s41577-019-01802019) 1-).
8 Pulko, V. et al. Human memory T cells with a naive phenotype accumulate with aging and respond to persistent viruses. Nat Immunol 17, 966-975, doi:10.1038/ni.3483 (2016).
9 Fang, F. et al. Expression of CD39 on Activated T Cells Impairs their Survival in Older Individuals. Cell Rep 14, 1218-1231, doi:10.1016/j.celrep.2016.01.002 (2016).
10 Canale, F. P. et al. CD39 Expression Defines Cell Exhaustion in Tumor-Infiltrating CD8 (+) T Cells. Cancer Res 78, 115-128, doi:10.1158/0008-5472.CAN-16-2682018) 4).
11 Aw, D. et al. Disorganization of the splenic microanatomy in ageing mice. Immunology 148, 92-101, doi:10.1111/imm.12590 (2016).
12 Slusarczyk, P. et al. Impaired iron recycling from erythrocytes is an early hallmark of aging. Elife 12, doi:10/7554.eLife.79196 (2023).
13 Gom, I. et al. Relationship between serum albumin level and aging in community-dwelling self-supported elderly population. J Nutr Sci Vitaminol (Tokyo) 53, 37-42, doi:10.3177/jnsv.53.37 (2007).

14 Jabara, H. H. et al. A missense mutation in TFRC, encoding transferrin receptor 1, causes combined immunodeficiency. Nat Genet 48, 74-78, doi:10.1038/ng.3465 (2016).
15 Frost, J. N. et al. Hepcidin-Mediated Hypoferremia Disrupts Immune Responses to Vaccination and Infection. Med (NY 179-164, 2 (e112, doi:10.1016/j.medj.2020.10.004 (2021).
16 Muckenthaler, M. U., Rivella, S., Hentze, M. W. & Galy, B. A Red Carpet for Iron Metabolism. Cell 168, 344-361, doi:10.1016/j.cell.2016.12.034 (2017).
17 Jin, J. et al. FOXO1 deficiency impairs proteostasis in aged T cells. Sci Adv 6, eaba1808, doi:10.1126/sciadv.aba1808 (2020).
18 Delpoux, A. et al. FOXO1 constrains activation and regulates senescence in CD8 T cells. Cell Rep 34, 108674, doi:10.1016/j.celrep.2020.108674 (2021).

What is claimed is:

1. A method of increasing proliferation of adaptive immune cells of a subject at least 65 years old, the method comprising contacting the immune cells with an agent which increases the level of labile iron in the immune cells and/or reduces toxicity of hemoglobin, heme or a degradation product thereof, thereby increasing the proliferation of the adaptive immune cells of the subject.

2. A method of treating or preventing an infectious disease or an inflammatory disease in a subject at least 65 years old, the method comprising administering to the subject a therapeutically effective amount of an agent which increases the level of labile iron in immune cells and/or reduces toxicity of hemoglobin, heme or a degradation product thereof, thereby treating or preventing the infectious disease or an inflammatory disease in the subject at least 65 years old.

3. A method of vaccinating a subject at least 65 years old, the method comprising administering to the subject a vaccine and an agent which increases the level of labile iron in immune cells and/or reduces toxicity of hemoglobin, heme or a degradation product thereof, thereby vaccinating the subject.

4. The method of claim 1, wherein said cells are lymphocytes.

5. The method of claim 4, wherein said lymphocytes are T lymphocytes.

6. The method of claim 4, wherein said lymphocytes are B lymphocytes.

7. The method of claim 3, wherein said vaccine is selected from the group consisting of a flu vaccine, a pneumococcal vaccine, a shingles vaccine, respiratory syncytial virus (RSV), and a tetanus-diptheria-pertussis vaccine (Tdap).

8. The method of claim 1, wherein said agent is at least one selected from the group consisting of:
(i) labile iron;
(ii) a scavenger of hemoglobin or heme or a degradation product thereof;
(iii) a lysosomal inducer; and
(iv) a heme crystal breaking agent.

9. The method of claim 8, wherein said labile iron is selected from the group consisting of ferric ammonium citrate (FAC), Ferrous sulfate, ferrous gluconate, ferrous ascorbate, ferrous fumarate, ferrous bisglycinate, iron polymaltose, iron sucrose and Iron Dextran.

10. The method of claim 8, wherein said scavenger is selected from the group consisting of hemopexin, heptoglobin and albumin.

11. The method of claim 8, wherein said lysosomal inducer is selected from the group consisting of ambroxol, gastrodin, a CDK4/6 inhibitor and an autophagy inducer.

12. The method of claim 11, wherein said CDK4/6 is selected from the group consisting of LY2835219 (e.g., Abemaciclib) and PD0332991 (e.g., palbociclib).

13. The method of claim 11, wherein said autophagy inducer is selected from the group consisting of rapamycin, torin1, metformin, resveratrol and spermidine.

14. The method of claim 8, wherein said heme crystal breaking agent is selected from the group consisting of quinine, chloroquine, amodiaquine, quinidine, quinacrine, halofantrine and mefloquine.

15. The method of claim 1, wherein said agent is administered or formulated for administration in an acute manner (i.e., not more than 14 days).

16. The method of claim 8, wherein said scavenger is administered or formulated for administration in a chronic manner (i.e., more than 2 weeks).

17. The method of claim 1, wherein the subject is selected according to measuring a level of protein or mRNA of at least one stress gene selected from the group consisting of CD39, HO1, HBB-BS, HBA, HMOX1, HEBP1, BLVRB, CPOX, FTL1, HMBS and FTH1, wherein a level above a predetermined threshold (e.g., higher than that of a young subject below 65 years old) is indicative of suitability to the treatment.

18. The method of claim 17, wherein said level is a peripheral blood level.


SEQUENCE LISTING (12 sequences)

SEQ ID NO: 1 - DNA, 20 bp, synthetic construct
  source 1..20: mol_type = other DNA
aagatcccgaacctctctct

SEQ ID NO: 2 - DNA, 22 bp, synthetic construct
  source 1..22: mol_type = other DNA
ttatcaaggctcccaagttctc

SEQ ID NO: 3 - DNA, 20 bp, synthetic construct
  source 1..20: mol_type = other DNA
aagctgtcatcgtgctactg

SEQ ID NO: 4 - DNA, 23 bp, synthetic construct
  source 1..23: mol_type = other DNA
cagttagtggttggtctcctatg

SEQ ID NO: 5 - DNA, 18 bp, synthetic construct
  source 1..18: mol_type = other DNA
tcaaccgccagatcaacc

SEQ ID NO: 6 - DNA, 19 bp, synthetic construct
  source 1..19: mol_type = other DNA
tcagtttctcggcatgctc

SEQ ID NO: 7 - DNA, 22 bp, synthetic construct
  source 1..22: mol_type = other DNA
cgtggatctgtgtcttgcttca

SEQ ID NO: 8 - DNA, 20 bp, synthetic construct
  source 1..20: mol_type = other DNA
gcgaagagacggtgcagact

SEQ ID NO: 9 - DNA, 21 bp, synthetic construct
  source 1..21: mol_type = other DNA
gttcaaacagctctatcgtgc

SEQ ID NO: 10 - DNA, 21 bp, synthetic construct
  source 1..21: mol_type = other DNA
tctttgtgttcctctgtcagc

SEQ ID NO: 11 - DNA, 21 bp, synthetic construct
  source 1..21: mol_type = other DNA
ccgccatgtctctagtgatcc

SEQ ID NO: 12 - DNA, 20 bp, synthetic construct
  source 1..20: mol_type = other DNA
ggtgaggtcgatgtctgctt